(19) United States
(12) Patent Application Publication
(10) Pub. No.: US 2017/0014070 A1
(43) Pub. Date: Jan. 19, 2017

(54) SLEEP SENSOR SYSTEM

(71) Applicant: ALAN HOLMES, Goleta, CA (US)

(72) Inventor: ALAN HOLMES, Goleta, CA (US)

(21) Appl. No.: 14/801,617

(22) Filed: Jul. 16, 2015

(51) Int. Cl. A61B 5/00; A61B 5/11

(52) U.S. Cl. CPC A61B 5/4815; A61B 5/11; A61B 5/1126; A61B 5/7203; A61B 5/7225; A61B 5/0022; A61B 5/742; A61B 5/7271; A61B 5/6887

(57) ABSTRACT

A system for monitoring sleep quality, as well as sensing movement in the proximity of the sensor, uses sensitive electronics to sense electric field fluctuations in its vicinity. Such fluctuations can be caused by, for example, cloth rubbing on skin or against bedsheets, or feet shuffling across a floor, all of which produce static charge. The static charge is not stable, since either the source is moving, or cloth is touching and pulling away from skin and altering the charge distribution. Even movement through the air of an object leads to charging of the object. A static charge sensor is connected to a microprocessor which detects and logs such charge fluctuations, and preferably transmits the data to a handheld computing device either using Bluetooth or wireless means. The hand-held device preferably analyzes the data to score the quality of a user's sleep, or to capture nearby movement of any entity.

### BACKGROUND OF THE INVENTION

Many individuals are troubled by a poor night's sleep. A common expression is that someone “tossed and turned all night long” to describe a sleepless night. However, it is difficult the next morning to put a quantitative estimate on how much sleep one actually experienced. Mack et al (U.S. Pat. No. 7,396,331) describes a system for measuring sleep quality using a variety of sensors, such as temperature, electromagnetic and vibration sensors. However, it is unlikely that these sensing methods have the sensitivity needed to provide an accurate determination of sleep quality.

There are many other sensor designs available which can measure sleep. Some devices can be worn on a wrist, and use accelerometers to determine when a person is moving. Some sensors may be attached to a head-band, and measure brain waves at night. Another possibility is sensors that attach around the chest to detect either a person's pulse rate or breathing. Such approaches require something attached to the body, which is undesirable to most individuals, and not an option for infants and small children for safety reasons. To avoid this severe limitation, sensors have been developed that attach to the mattress or bed frame, and measure vibration. However, sensitivity is compromised by such designs.

Another approach is to have a sensitive microphone next to the bed that measures snoring, or movement, but not everyone snores during a poor night's sleep. Perhaps the poor sleep is due to an aching back, or arthritic leg, in which case no snoring is involved. The sensitivity of such a device to movement is limited.

Movement in bed is not the only aspect of sleep that can be considered. Sleepwalking, particularly in children, is not uncommon. A device that can determine if a child left the bed during the night is desirable. A similar consideration applies to elderly individuals, who often suffer from poor sleep patterns and roam the house at night, but have little memory of it in the morning. In these cases, a caregiver needs some way of ascertaining the severity of the problem to determine if remedial steps need to be applied. Of course, a video camera could be set up and left to run all night, filming the subject, but this approach yields hours of recording to inspect, a tedious task only useful for capturing large movements.

### SUMMARY OF THE INVENTION

The present sleep sensor system uses an electric field sensor, preferably constructed using a sensitive electrometer amplifier connected to a double-sided circuit board. One side of the circuit board is used as a sensing reference, and the other as the sensing electrode. The circuit board will typically be 10 to 40 square inches in size, and 0.06 inches thick. When the electric field in the area changes, charge will be pulled into or forced out of the sensing electrode, and flows into the electrometer, which is preferably a trans-impedance amplifier that converts the charge to a voltage. The electrometer amplifier is preferably an integrated circuit (IC) having high input impedance, enabling the use of high impedance feedback resistors for increased sensitivity. After the charge is converted to a voltage, it is low-pass filtered to remove noise caused by the 50/60 Hz voltage in the building wiring, and further amplified. An analog-to-digital (A/D) converter converts this voltage to a digital number which is recorded in memory, preferably by a microprocessor. At some later time a user can establish a link with the device and download the data. In general the link can either be wireless or Bluetooth, or a similar technology.

The motion of a person across or within the bedsheets at night generates static charges which are pulled apart by the person's motion, producing an electric field. The electric field sensor senses the changes in the electric field proximate to the sensor, and logs the data all night long. In the morning the user can interrogate the sensor and determine the fraction of the night when he or she was still, which is a good indication of sleep.

Another use for the present sensor is capturing movement of an entity of any kind at any desired location. Many people have experienced the surprise of finding a light on in the morning, a door to the outside left open, or something in the house out of place. This device could provide confirmation to the user that he or she is not merely getting forgetful, but that something or someone is actually moving about the house at night. The device has a very high sensitivity; even rodents moving around in the vicinity can be detected.

As noted above, the device preferably includes a logging capability that can save the data and display it to the user graphically on either a computer or hand-held device such as a smart phone or tablet. Multiple sensing devices might be placed about a house, each of which can be read out and used to detect movement occurring within the house at night.

These and other features, aspects, and advantages of the present invention will become better understood with reference to the following description and claims.

### BRIEF DESCRIPTION OF THE DRAWINGS:

FIG. 1 is a block diagram illustrating one possible embodiment of the present sleep sensor system.

FIG. 2 is a block diagram illustrating how the present sensor system can be read out over a wireless link.

FIG. 3 is a diagram illustrating how the entire sensor system can be mounted into a picture frame so as to be unobtrusively employed in a user's house.

FIG. 4 illustrates a recording of an actual night's sleep.

### DESCRIPTION OF THE PREFERRED EMBODIMENT

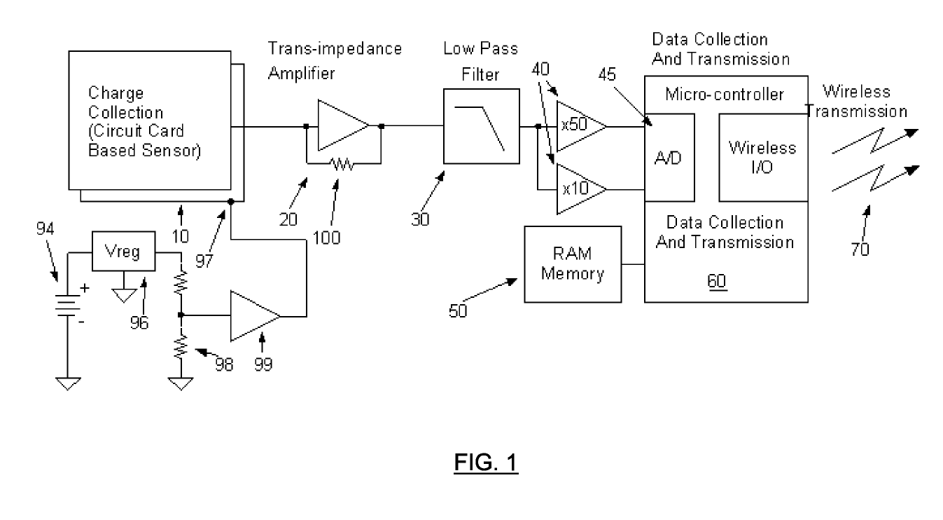

A block diagram of one possible embodiment of the present sleep sensor system is shown in FIG. 1. As noted above, the motion of a person across or within bedsheets at night, feet shuffling across a floor, etc., generate static charges, producing a fluctuating electric field. An electric field sensor senses changes in the electric field proximate to the sensor, which here consists of a double-sided circuit board 10. One side of circuit board 10 is used as a sensing reference (the “reference plate”), and the other as the sensing electrode (the “sensing plate”). The charge induced by the varying electric field is converted to a voltage by an amplifier 20, preferably a trans-impedance amplifier. The amplifier output is low-pass filtered by circuitry 30 to suppress noise induced by the 50/60 Hz field present due to house wiring; a typical rolloff frequency would be around 1 Hz. One or more gain stages 40 follow filter 30, preferably with two levels of gain (here, ×10 and ×50) that are user-selectable. The signal is then digitized by an A/D converter 45, which may be a discrete device or part of a micro-controller 60. The converted signal is then saved into memory 50, preferably RAM. The stored data can be regularly conveyed to another device with a user interface via a wireless transmitter 70 such as a Bluetooth transmitter, or the user can request the data over wireless link 70.

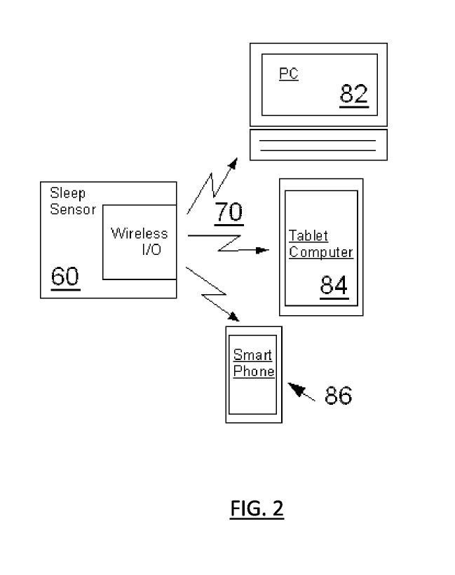

FIG. 2 illustrates how the stored data can be read out over a wireless link 70 by, for example, a PC (or MAC) computer 82, a tablet computer 84, or smartphone 86. Software can be created for use on one or more of these devices to receive the data and present it in a user-friendly form. For example, the software could apply a simple algorithm which determines the number of minutes during a night for which the user did not move for 3 minutes or longer (as detected by the sensor), and sum these minutes together. This could be compared to the number of minutes between the obvious events of the user getting into bed or out of bed to determine a percentage of time asleep; the numbers of minutes and the calculated percentage could then be displayed to the user.

As noted above, circuit board 10 is preferably double-sided, 10 to 40 square inches in size, and 0.06 inches thick. Each side of the board is preferably copper-clad, with the copper cladding on one side serving as the sensing electrode and the cladding on the other side serving as a sensing reference The shape of the board is not particularly important, and could be customized to allow disguising it in other common objects, such as a book.

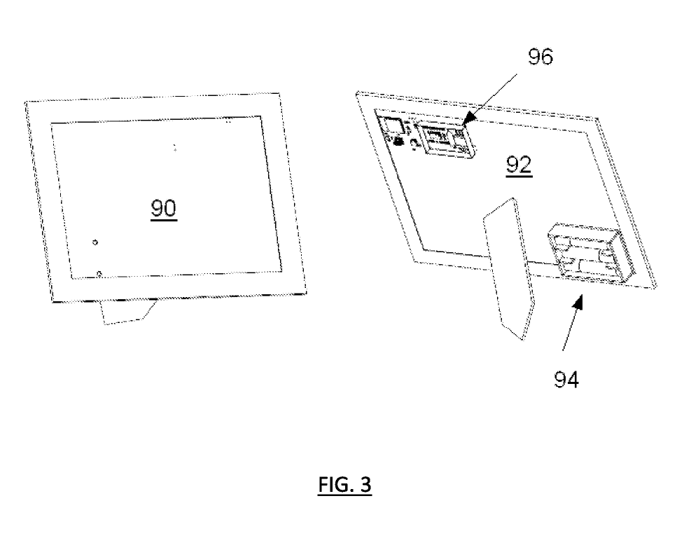

The electric field sensor and associated electronics can be implemented in many different ways; the following describes a preferred embodiment. The sensor and electronics are arranged on a 5 by 7 inch circuit board, which mounts into an object such as a standard picture frame; this is illustrated in FIG. 3. The user can mount a picture to the front side 90 of the frame, typically under glass, so that from the front side of the device it simply appears to be a picture in a frame. The back side 92 of the frame holds a power source such as batteries 94, which power the electric field sensor and associated electronics 96. The sensor's active area is preferably 28 square inches of the total circuit board area of 35 square inches. Referring back to FIG. 1, a virtual ground 97 is preferably created using a 3.3 volt linear voltage regulator 96 from the +lead of the batteries 94 followed by a simple resistor divider 98, and followed by a buffer amplifier 99. One side of double-sided copper clad board 10 is connected to this virtual ground to form the reference plate, against which all electrical fields are measured. The other side of the copper-clad board is the sensing plate. Charge induced in the sensing plate by a fluctuating external electric field flows into the trans-impedance amplifier 20, preferably constructed using a Max406BCPA Single Supply OpAmp with a 1 GΩ feedback resistor 100. The amplified signal is then low-pass filtered with a 3 dB cutoff frequency of 1 Hz. This is necessary since the building wiring produces a large 50/60 Hz signal which can degrade the system's sensitivity if not filtered as described. The amplified signal is preferably further amplified by either 10×, or 50×, depending on user configuration, and the signal digitized and saved in a BlueGiga Low Energy Single Mode micro-controller module which implements a Bluetooth link. A user, using either an iPad, iPhone, or Android device supporting the Bluetooth Low Energy protocol, can then communicate with the BlueGiga device and download the data for display to the user. The user can also preferably configure the data collection parameters such as the gain, seconds per sample, and number of samples.

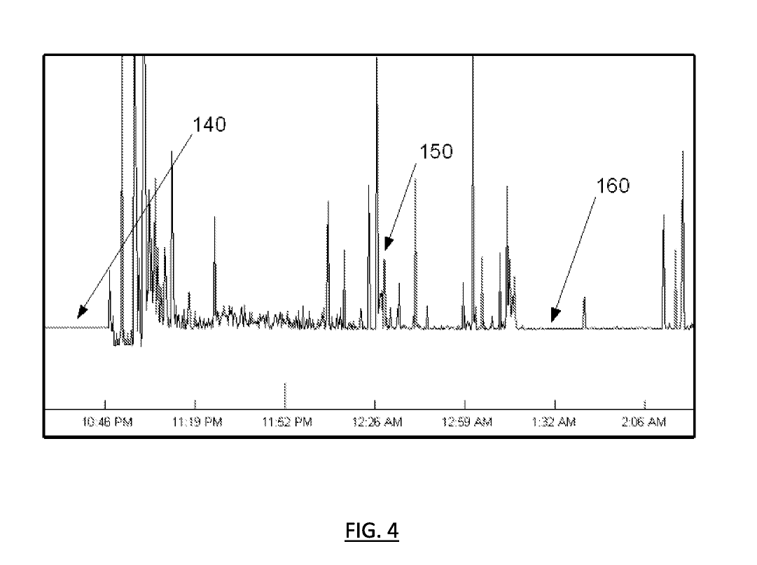

The data downloaded from the sensor can be presented in graphical form, as illustrated in FIG. 4, or processed to yield a metric for the sleep quality. In FIG. 4, segment 140 shows the low noise level recorded by the sensor before the sleeper enters the room. Moments of large movement 150 indicate a restless sleeper, and quiet segments indicate good sleep 160. The data can be processed in many different ways; the metric currently used for sleep quality is calculated as follows. The unit is set up to record data every 20 seconds using an aggregation mode to preserve the important information but reduce the amount of data required to be stored. It does this by the sampling the electric field every 1 second, and collecting 20 samples in temporary memory until 20 seconds have passed. At that time it constructs and logs a 16 bit number that is the average of the 20 samples, summed with the difference of the maximum and minimum values recorded for the 20 samples. So, any movement recorded in the 20 second interval is captured, as well as offset shifts, which might be real or indicate a hardware problem. When the data samples are downloaded to the iPad, Android, or iPhone hand-held device, the wireless display device uses application software installed on it to download previously-stored data samples and analyze the stored data. The application software allows the previous night's electric field data to be parsed to determine when an individual entered and left the bed. That number of minutes is a metric displayed on the device as the total number of minutes in bed, referred to as the BED-TIME.

The next metric calculated is the number of minutes during the BED-TIME where no movement has occurred; this metric is called STILL-TIME. STILL-TIME is calculated as the total time where the sensor output signal does not exceed a THRESHOLD data value set above a baseline. The baseline is the signal level seen in nearby quiet time periods when the sleeper has not moved. However, a better metric of sleep is obtained if one assumes STILL-TIME does not count as sleep until at least three minutes have passed since the last motion of the sleeper, three minutes being called the QUIET-INTERVAL. So, the number of minutes in periods where no movement has occurred for at least the QUIET-INTERVAL or longer is summed to yield the SLEEP-TIME. Of course, these parameters, THRESHOLD or QUIET-INTERVAL, can be customized to an individual's situation. As an example, typical numbers for these parameters might be 540 minutes for BED-TIME, 460 minutes for STILL-TIME, and 360 minutes for SLEEP-TIME. Experience to date indicates that only about 66% of the time in bed is actually time asleep.

The embodiments of the invention described herein are exemplary and numerous modifications, variations and rearrangements can be readily envisioned to achieve substantially equivalent results, all of which are intended to be embraced within the spirit and scope of the invention as defined in the appended claims.

I claim:

1. A system that detects movement, comprising:
an electric field sensor which produces an output that varies with the electric field proximate to said sensor;
circuitry coupled to receive the output of said electric field sensor and arranged to produce and store digital data which represents said electric field sensor output over time; and
a wireless transmitter arranged to wirelessly transmit said stored digital data.

2. The system of claim 1, wherein said electric field sensor comprises a double-sided copper-clad circuit board, in which one side of said circuit board operates as a sensing electrode and the other side of said circuit board operates as a sensing reference.

3. The system of claim 2, further comprising a virtual ground coupled to the sensing reference side of said circuit board.

4. The system of claim 3, further comprising:
a supply voltage and a circuit ground;
a voltage regulator connected to said supply voltage and providing a regulated voltage at an output;
a voltage divider connected between said regulated voltage and said circuit ground; and
a buffer amplifier arranged to buffer the output of said voltage divider, the output of said buffer amplifier providing said virtual ground.

5. The system of claim 4, wherein said voltage regulator is a 3.3 volt linear voltage regulator.

6. The system of claim 1, wherein said circuitry comprises:
an amplifier connected to receive the output of said electric field sensor;
a low pass filter arranged to filter out noise due to 50 Hz/60 Hz house wiring;
an analog-to-digital converter which provides a digital output that represents the output of said electric field sensor; and
a storage means arranged to receive and store the output of said analog-to-digital converter.

7. The system of claim 6, wherein said amplifier is a trans-impedance amplifier.

8. The system of claim 6, further comprising one or more gain stages arranged to amplify the output of said amplifier.

9. The system of claim 8, wherein said one or more gain stages comprises at least two gain stages having different gains, the connection of one of said gain stages to said amplifier output being user-selectable.

10. The system of claim 1, wherein said wireless transmitter is a Bluetooth transmitter.

11. The system of claim 1, further comprising a device arranged to receive, process and graphically display said wirelessly transmitted data.

12. The system of claim 11, wherein said device is an iPhone, Android tablet, an iPad or a personal computer.

13. The system of claim 1, wherein said circuitry is arranged to:
periodically sample the output of said electric field sensor;
store a fixed number of said sampled values;
calculate a value which represents the average of said fixed number of sample values;
sum said calculated value with the minimum value and the maximum value of said fixed number of sampled values; and
store said summed values to be transmitted by said wireless transmitter.

14. The system of claim 13, further comprising a device arranged to receive, process and graphically display data based on said wirelessly transmitted summed values, said device arranged to, based on said summed values:
derive a total number of minutes that the user was in bed; and
determine the number of said total minutes during which no movement was detected.

15. The system of claim 14, further comprising:
establishing a threshold value for detecting movement; and
detecting lack of movement only when said summed values are below said threshold value.

16. The system of claim 15, wherein said threshold value can be set by a user.

17. The system of claim 14, further comprising:
establishing a time interval; and
determining the number of consecutive minutes during which no movement was detected which exceeds said time interval; and
summing said consecutive minutes together.

18. The system of claim 17, wherein said time interval can be set by a user.

19. The system of claim 17, further comprising displaying said summed number of consecutive minutes.

20. The system of claim 1, further comprising an object upon which said sensor, circuitry and wireless transmitter are mounted.

21. The system of claim 20, wherein said object is a picture frame.
